(19) United States
(12) Patent Application Publication
(10) Pub. No.: US 2024/0426798 A1
(43) Pub. Date: Dec. 26, 2024

(54) A TOOL FOR DETERMINING THE BEE SPECIES ORIGIN OF HONEY PRODUCTS IN THE PHILIPPINES

(71) Applicant: Department of Science and Technology - Philippine Nuclear Research Institute (DOST-PNRI), Quezon City (PH)

(72) Inventors: Angel T. Bautista, VII, Quezon City (PH); Marco R. Lao, Quezon City (PH); Norman DS. Mendoza, Quezon City (PH); Roland V. Rallos, Quezon City (PH); Cleofas R. Cervancia, Quezon City (PH)

(21) Appl. No.: 17/609,908

(22) PCT Filed: May 12, 2021
(86) PCT No.: PCT/PH2021/050010

§ 371 Date: Nov. 9, 2021

(30) Foreign Application Priority Data
May 11, 2021 (PH) 1-2021-050218

(51) Int. Cl. G01N 33/02; G06F 17/18

(52) U.S. Cl. CPC G01N 33/02; G06F 17/18

(57) ABSTRACT

In the Philippines, the Department of Agriculture-Bureau of Agricultural Research (DA-BAR) identifies honey production as one of the most promising small and medium enterprises. Stingless bee honey, in particular, has excellent market potential for the food, pharmaceutical, and cosmetic industries due to its positive health effects. Several studies show that stingless bee honey has therapeutic properties that are at par or even significantly better than honey produced by A. mellifera, which includes the Manuka Honey. In fact, stingless bee honey is being regarded in the ASEAN Region as the next ‘super food’ industry, that can be a competitor to the Manuka Honey. Therefore, there is a need for a tool to be able to determine which species of bee produced a particular honey product. With this need as motivation, the proponents of the technology developed a tool for determining the bee species origin of honey products in the Philippines.

### TECHNICAL FIELD OF THE INVENTION

This invention relates generally to tools or methods specially adapted for use with animals.

### BACKGROUND OF THE INVENTION

Honey is a natural sweet substance produced by bees from plant nectar or excretions of plant-sucking insects on the living parts of plants. Bees collect this nectar or excretions and transform them by combing specific substances of their own, then deposit, dehydrate, and store in the honeycomb (Codex Stan, 1981). Honey has numerous documented nutritional and therapeutic benefits such as anti-microbial, antioxidant, anti-tumor, antidiabetic, and wound-healing properties (Eteraf-Oskouei & Najafi, 2013; Mandal & Mandal, 2011; Ullah et al., 2018; Amin et al., 2018). These benefits put a high demand for honey in the market but also made honey a subject for adulteration, a serious worldwide problem that has been damaging the beekeeping industry as well as risking the health of consumers. The Philippines, being a tropical country, is home to diverse species of social and solitary bees that contribute to agricultural productivity and biodiversity through pollination. Of the nine known honeybees in the world, five are indigenous to the Philippines, namely Apis cerana, Apis dorsata, Apis breviligula, Apis andreniformis, and Apis nigrocincta (Cervancia, 2018). In terms of honey supply, the introduced European honeybee Apis mellifera is still the primary producer of honey in the Philippines. The share of A. mellifera, A. cerana, combined A. breviligula and dorsata, and T. biroi in the Philippine honey supply are 69.2%, 2.3%, 24.8%, and 3.7%, respectively (Cervancia and Fajardo, 2012). A better understanding of the behavior of the native bee species A. cerana, A.breviligua, and A. dorsata and improved management practices have led to an increase in their share to the honey supply. Domestication of stingless bees, particularly Tetragonula biroi, has also gained more and more widespread for its utility in large scale pollination (Cervancia, 2018; Heard, 1999) and the therapeutic and nutritional values of its honey, pollen and propolis (Belina-Aldemita, Opper, Schreiner, & D'Amico, 2019; Cumbao et al., 2016; Sahlan et al., 2019). In the Philippines, the Department of Agriculture-Bureau of Agricultural Research (DA-BAR) identifies honey production as one of the most promising small and medium enterprises. An agricultural farmer can make an additional P100,000 to P400,000 per year by keeping bee colonies beside their farms (Business World, 2012). In 2006, the Beekeepers Association of the Philippines, Inc. (BAPI), one of the largest beekeeper associations in the Philippines, estimated the total value of honey (both produced during this year and those in stock) in the country to be around P90 million (Esplana, 2009). Additionally, the Philippines import tons and tons of honey from other countries. In January-May 2017, the Philippines imported around P24 million worth of honey from the US alone (USDA, 2017). Stingless bee honey, in particular, has excellent market potential for the food, pharmaceutical, and cosmetic industries due to its positive health effects. Several studies show that stingless bee honey has therapeutic properties (i.e., antidiabetic, wound healing, anticancer, fertility, and treatment for eye diseases and hypertension) that are at par or even significantly better than honey produced by A. mellifera (European Honey, the most common type), which includes the world-famous Manuka Honey (Amin et al., 2018; Ávila et al., 2018). In fact, stingless bee honey is being regarded in the ASEAN Region as the next ‘super food’ industry, that can be a competitor to the world-famous Manuka Honey (Hamid, 2019; Tay, 2017). Therefore, there is a need for a tool to be able to determine and certify which species of bee produced a particular honey product.

Zhou et al., 2018 studied the authenticity and geographic origin of global honeys determined using carbon isotope ratios and trace elements. The findings show the common and prevalent issues of honey authenticity and the mislabeling of its geographic origin can be identified using a combination of stable carbon isotopes and trace element concentrations.

Dinca et al., 2014 investigated the Geographical and Botanical Origin Discrimination of Romanian Honey Using Complex Stable Isotope Data and Chemometrics. In this study, stable isotopes selected as representative discrimination parameters of different botanical or geographical origin were determined in 40 honey samples using isotope ratio mass spectrometry (IRMS) and site specific natural isotopic fractionation measured by nuclear magnetic resonance (SNIF-NMR) methods.

Berriel et al., 2019 discussed the use of a mathematical expression, the logistic regression (LR), in differentiating eucalyptus honey from pasture honey in Uruguay. The study reveals a correct classification of total honey samples at 89.9%. The researchers concluded that the LR model is more accurate compared to Linear Discriminant Analyses (LDA).

Schellenberg et al., 2009 studied the Multielement stable isotope ratios (H, C, N, S) of honey from different European regions. The results show that the stable isotope ratios of the four bio-elements carbon, nitrogen, hydrogen and sulfur in honey protein can be applied to verify the origin of honey. Carbon and sulfur were identified by canonical discriminant analysis as providing the maximum discrimination between honey samples. For seven regions the percentage of correct classified samples is greater than 70%. It was concluded that the methodology in its current state can be used to provide reliable origin information.

ES2688968A1 relates to the detection of adulteration and identification of the geographical origin of honey. The invention relates, in general, to a method for the analysis of honey, the detection of adulteration and origin by means of a portable system based on the laser-induced plasma spectroscopy (LIBS) technique. In particular, it refers to a method of 10 honeys for detection of adulterations based on the intensity relationships of emission lines of elements present in honey, including pure honeys or mixtures of honeys of different origin and identification, for example, for the fraud detection.

### SUMMARY OF THE INVENTION

This invention provides a tool for determining the bee species origin of honey products in the Philippines.

### DETAILED DESCRIPTION OF THE INVENTION

A tool for determining the bee species origin of honey products in the Philippines was developed. Briefly, this tool is capable of determining or certifying which bee species (i.e., between A. mellifera, A. breviligula, and stingless bees or T. biroi; only between three species for now) produced a honey product. The tool is comprised of the following components: First, is a Database, which is a unique tool database by collecting 36 authentic honey products (13 from A. mellifera, 13 from A. breviligula, and 10 from stingless bees) from various locations in the Philippines, and analyzing these honey samples for eleven (11) parameters. The eleven (11) parameters are a. δ13C of the bulk honey (B.d13C, in per mille); b. δ13C of proteins extracted from honey (P.d13C, in per mille); c. Difference of B.d13C and P.d13C (B-P.d13C, in per mille); d. δ15N of proteins extracted from honey (P.d15N, in per mille); e. δ2H of water extracted from honey (d2H, in per mille); f. δ18O of water extracted from honey (d18O, in per mille); g. Percent carbon content (% C) of honey (PC, in percent); h. Percent nitrogen content (% N) of honey (PN, in percent); i. Percent moisture content of honey (Moisture, in percent); j. Percent ash content of honey (Ash, in percent); and k. Percent reducing sugar content of honey (Reducing Sugar, in percent).

TABLE 1
The complete tool database of the disclosure.

| | | B- | | | | | | | | Reducing | |
|---|---|---|---|---|---|---|---|---|---|---|---|
| B.d13C | P.d13C | P.d13C | P.d15N | PC | PN | d2H | d18O | Moisture | Ash | Sugar | Species |
| −24.73 | −24.54 | −0.19 | 4.31 | 33.8 | 11 | −42.88 | −0.15 | 20.29 | 0.23 | 71.03 | Breviligula |
| −25.48 | −25.44 | −0.04 | 2.9 | 31 | 10.1 | −53.05 | −1.46 | 17.86 | 0.14 | 70.78 | Breviligula |
| −27.13 | −26.25 | −0.88 | −1.92 | 33.9 | 12.4 | −31.67 | 1.5 | 19.25 | 0.44 | 68.71 | Breviligula |
| −26.85 | −26.37 | −0.48 | 4.24 | 32.1 | 11 | −35.64 | 0.36 | 21.42 | 0.38 | 70.98 | Breviligula |
| −25.4 | −24.98 | −0.41 | 3.02 | 32 | 9.8 | −38.1 | 1.55 | 16.62 | 0.32 | 67.86 | Breviligula |
| −29.24 | −28.36 | −0.41 | 2.27 | 34.9 | 13 | −95.46 | −9.09 | 18.44 | 0.4 | 69.49 | Mellifera |
| −24.42 | −24.14 | −0.28 | 3.27 | 31.5 | 10.7 | −40.1 | −0.36 | 17.74 | 0.07 | 73.66 | Mellifera |
| −26.28 | −25.55 | −0.73 | 3.27 | 30.1 | 12.6 | −12.17 | −1.28 | 14.13 | 0.1 | 76.01 | Mellifera |
| −27.8 | −27.53 | −0.27 | 0.26 | 39.8 | 12.2 | −68.75 | −3.82 | 23.19 | 1.01 | 39.81 | Stingless |
| −25.31 | −25.76 | 0.45 | 3.12 | 38.4 | 12.5 | −41.29 | −2.16 | 18.31 | 1.24 | 50.16 | Stingless |
| −25.3 | −25.34 | 0.04 | 2.97 | 39.6 | 11.9 | −43.73 | 0.11 | 18.69 | 1.31 | 47.56 | Stingless |
| −26.05 | −26.47 | 0.42 | −0.79 | 36.3 | 8.4 | −34.84 | −0.73 | 19.01 | 0.3 | 71.17 | Breviligula |
| −26.13 | −26.25 | 0.12 | 0.03 | 32 | 10.4 | −32.93 | −1.25 | 23.11 | 0.22 | 60.89 | Stingless |
| −23.79 | −23.45 | −0.34 | 3.13 | 30.1 | 11.4 | −29.63 | 0.3 | 12.94 | 0.07 | 73.08 | Mellifera |
| −29.57 | −27.61 | −1.96 | −2.31 | 31.3 | 9.2 | −33.93 | −2.09 | 22.75 | 0.29 | 66.29 | Breviligula |
| −24.96 | −25.01 | 0.05 | 4 | 34.7 | 10.8 | −5.78 | 1.2 | 17.44 | 0.61 | 56.21 | Stingless |
| −25.56 | −25.63 | 0.07 | −0.13 | 43.7 | 8.8 | −17.46 | −0.27 | 21.42 | 0.84 | 53.04 | Stingless |
| −26.04 | −26.22 | 0.18 | 1.35 | 39.5 | 8.8 | −20.51 | −1.41 | 20.24 | 0.69 | 58.67 | Stingless |
| −26.24 | −26.14 | −0.1 | −2.22 | 34.7 | 10.4 | −34.06 | −3.8 | 19.74 | 0.25 | 70.72 | Breviligula |
| −25.55 | −25.69 | 0.14 | −1.56 | 36.9 | 9.7 | −27.12 | −0.76 | 17.14 | 0.3 | 73.72 | Breviligula |
| −23.16 | −24.79 | 1.62 | 6.86 | 31.6 | 10.3 | −35.28 | −6.25 | 12.47 | 0.36 | 71.27 | Mellifera |
| −25.95 | −26.25 | 0.3 | 1.47 | 40.4 | 8.8 | −31.75 | −2.09 | 18.08 | 0.63 | 52.04 | Stingless |
| −27.2 | −25.57 | −1.63 | 2.22 | 36.3 | 12.5 | −38.72 | 0.68 | 18.44 | 1.45 | 66.07 | Breviligula |
| −26.17 | −25.82 | −0.36 | −2.24 | 33.9 | 12 | −26 | 0.32 | 19.89 | 0.45 | 69.29 | Breviligula |
| −24.31 | −23.92 | −0.39 | 3.78 | 32.1 | 10.4 | −23.98 | 3.84 | 12.89 | 0.07 | 76.89 | Mellifera |
| −28.87 | −26.23 | −2.64 | 1.79 | 33.2 | 11.1 | −22.87 | −1.27 | 13 | 0.11 | 80.05 | Mellifera |
| −25.09 | −26.53 | 1.44 | 1.49 | 33 | 10.2 | −66.3 | −4.05 | 14.57 | 0.2 | 79.67 | Mellifera |
| −27.13 | −25.77 | −1.36 | 0.25 | 39 | 11.9 | −55.12 | −4.28 | 22.64 | 0.83 | 60.68 | Stingless |
| −28.64 | −26.13 | −2.51 | 3.54 | 30.7 | 10.9 | −30.81 | −2.18 | 14.73 | 0.08 | 75.79 | Mellifera |
| −27.97 | −26.83 | −1.13 | −1.62 | 33.6 | 10.3 | −37.76 | −4.19 | 20.11 | 0.42 | 69.03 | Breviligula |
| −28.81 | −23.84 | −4.96 | 2.65 | 30.3 | 10.2 | −55.03 | −4.3 | 15.8 | 0.05 | 79.02 | Mellifera |
| −25.75 | −25.36 | −0.39 | 0.86 | 34.8 | 11.3 | −27.42 | −1.67 | 18.94 | 0.74 | 75.2 | Breviligula |
| −29 | −26.42 | −2.58 | 1.76 | 29.6 | 10 | −32.86 | −2.91 | 13.64 | 0.05 | 80.09 | Mellifera |
| −26.33 | −24.97 | −1.36 | 3.56 | 40.1 | 8.6 | −36.04 | −1.12 | 20.22 | 0.89 | 58.61 | Stingless |
| −25.25 | −25.28 | 0.03 | 2.27 | 35.2 | 10.2 | −36.76 | −0.57 | 13.22 | 0.24 | 73.78 | Mellifera |
| −26.32 | −25.32 | −1.01 | 3.04 | 30.2 | 10.8 | −28.49 | 0.48 | 15.1 | 0.06 | 77.12 | Mellifera |

Table 1 shows the complete tool database created using the technology of the disclosure. The tool database is composed of data and values from the 36 authentic honey products samples from the various locations in the Philippines and were analyzed using the eleven (11) parameters which were previously stated.

Parameters (a) to (c), (f), and (g) were analyzed using an elemental-analyzer-isotope ratio mass spectrometer (EA-IRMS) with sample preparation procedures following the Association of Official Analytical Chemists or AOAC method 998.12. Parameters (d) and (e) were likewise analyzed using IRMS, with the water from honey being extracted via cryogenic vacuum distillation. Parameter (h) was analyzed using the oven method following AOAC method 925.45B. Parameter (i) was analyzed using the gravimetric method following AOAC method 923.03. Parameter (j) was analyzed through the dinitrosalicylic acid (DNS) assay (Miller, 1959).

The second component of the tool are Equations of the two discriminant functions using the tool database constructed and described above, Linear Discriminant Analysis or LDA was performed to determine the equations of the two discriminant functions. LDA is a statistical method for classification that uses linear combinations (i.e., equations of the discriminant functions) of a set of parameters (i.e., the three parameters contained in the database) to predict which group an observation belongs to (i.e., which geographical region they were produced; Venables and Ripley, 2002). Furthermore, it is needed to select an optimal subset of the 11 parameters, that will have the highest accuracy in its predictive capabilities. To do this, we used four criteria indices: ccr21 (a function of the Roy first root test statistic; Roy, 1939), Tau2 or τ2 (a function of the Wilks' Lambda statistic; Wilks, 1932), Xi2 or ξ2 (a function of the Bartlett-Pillai test statistic; Pillai, 1955), and Zeta2 or ζ2 (a function of the Lawley-Hotelling trace test statistic; Hotelling, 1951). Using these criteria indices, we determined that the equations of the optimal subsets of the two discriminant functions are the following:

$$Full version ( 6 parameters ) :$$

$$LD 1 = 0.11815 * B . d 13 C - 0.00895 * P . d 15 N + 0.16533 * PC + 0.1602 * d 18 O + 0.35441 * Moisture - 0.14997 * Reducing . Sugar$$

$$LD 2 = 0.19871 * B . d 13 C - 0.23877 * P . d 15 N - 0.0588 * PC + 0.16066 * d 18 O + 0.38921 * Moisture + 0.11104 * Reducing . Sugar$$

$$Lite version ( 3 parameters ) :$$

$$LD 1 = - 0.16505 * d 18 O - 0.31322 * Moisture \text{} + 0.18909 * Reducing . Sugar$$

$$LD 2 = - 0.21058 * d 18 O - 0.46893 * Moisture - 0.13293 * Reducing . Sugar$$

There are two versions of the tool: the Full and Lite versions. The Full version requires data of 6 parameters (B.d13C, P.d15N, PC, d18O, Moisture, and Reducing.Sugar), while the Lite version requires data of 3 parameters (d18O, Moisture, and Reducing Sugar). The advantage of the Full version is that the accuracy of its predictive capability is higher than the Lite version (i.e., 94.4% for Full version vs. 92% for the Lite version). On the other hand, the advantage of the Lite version is that the user needs to supply information of only 3 parameters (versus the Full version's 6 parameters). The accuracies of the versions were determined using the Leave-One-Out cross-validation method for LDA (Table 2).

TABLE 2
Accuracies of the Full and Lite versions based on Leave-
One-Out cross validation method of the tool database.

| | | | | Accuracy |
|---|---|---|---|---|
| | | | | Total |
| | Prediction (Full version) | | | Accuracy = 94.4% |
| Reference | A. breviligula | A. mellifera | Stingless Bees | (34 out of 36) |
| A. breviligula | 13 | 0 | 0 | 100% |
| A. mellifera | 1 | 12 | 0 | 92.4% |
| Stingless Bees | 1 | 0 | 9 | 90.0% |
| | | | | Accuracy |
| | | | | Total |
| | Prediction (Lite version) | | | Accuracy = 91.7% |
| Reference | A. breviligula | A. mellifera | Stingless Bees | (33 out of 36) |
| A. breviligula | 13 | 0 | 0 | 100% |
| A. mellifera | 1 | 12 | 0 | 92.4% |
| Stingless Bees | 2 | 0 | 8 | 80.0% |

The third component is prediction or classification tool using the database and equations described above, the tool can now determine where the honey product was likely produced. First, the user needs to put in the values either of the 3 or 6 parameters and these values are obtained by having their honey samples analyzed for these parameters in capable laboratories depending on whether the user prefers to use the Full or Lite versions, respectively. Subsequently, the tool is executed, and its output shows the probability of the honey sample being produced either by A. breviligula, A. mellifera, or stingless bees.

1. A tool for determining the bee species origin of honey products in the Philippines, comprising the steps of:
a. collecting authentic honey products;
b. building the isotopic fingerprint database by analyzing honey products for various parameters;
c. performing Linear Discriminant Analysis (LDA);
d. determining the equations of the two discriminant functions;
e. determining the accuracy of the tool;
f. putting in the values of the parameters to the tool; and
g. executing the tool.

2. The process according to claim 1, wherein said parameters are δ13C of the bulk honey (B.d13C, in per mille); δ13C of proteins extracted from honey (P.d13C, in per mille); Difference of B.d13C and P.d13C (B-P.d13C, in per mille); δ15N of proteins extracted from honey (P.d15N, in per mille); δ2H of water extracted from honey (d2H, in per mille); δ18O of water extracted from honey (d18O, in per mille); Percent carbon content (% C) of honey (PC, in percent); Percent nitrogen content (% N) of honey (PN, in percent); Percent moisture content of honey (Moisture, in percent); Percent ash content of honey (Ash, in percent); and Percent reducing sugar content of honey (Reducing.Sugar, in percent).

3. The process according to claim 1, wherein said equations of the two discriminant functions for full version are LD1=0.11815*B.d13C−0.00895*P.d15N+0.16533*PC+0.16020*d18O+0.35441*Moisture−0.14997*Reducing.Sugar and LD2=0.19871*B.d13C−0.23877*P.d15N−0.05880*PC+0.16066*d18O+0.38921*Moisture+0.11104*Reducing.Sugar.

4. The process according to claim 1, wherein said equations of the two discriminant functions for lite version are LD1=−0.16505*d18O−0.31322*Moisture+0.18909*Reducing.Sugar and LD2=−0.21058*d18O−0.46893*Moisture−0.13293*Reducing.Sugar.

5. The process according to claim 1, wherein said accuracy of the tool is determined by Leave-One-Out cross-validation method.

6. The process according to claim 1, wherein said tool is executed within the platform of the R software.

7. A tool obtained by the process according to claim 1, wherein components are database, equations of the two discriminant functions, and prediction or classification tool.
